(19) United States
(12) Patent Application Publication
(10) Pub. No.: US 2007/0255196 A1
(43) Pub. Date: Nov. 1, 2007

(54) ULTRASONIC LIQUEFACTION METHOD AND APPARATUS USING A TAPERED ULTRASONIC TIP

(76) Inventor: David G. Wuchinich, Yonkers, NY (US)

Correspondence Address: Connolly Bove Lodge & Hutz LLP, P.O. Box 2207, Wilmington, DE, 19899-2207, US

(21) Appl. No.: 11/608,975

(22) Filed: Dec. 11, 2006

Related U.S. Application Data
(60) Provisional application No. 60/793,083, filed on Apr. 19, 2006.

(51) Int. Cl. A61B 17/20

(52) U.S. Cl. 604/22

(57) ABSTRACT

Ultrasonic vitreous liquefaction is achieved through use of an apparatus which includes a transducer for generating an ultrasonic vibration to longitudinally ultrasonically vibrate a solid ultrasonic tip. The tip is preferably tapered and the free end of the tip is preferably at least partially surrounded by a stationary sheath. The apparatus may include structure for applying irrigation and aspiration.

### CROSS REFERENCE TO RELATED APPLICATION

This application is based upon provisional application Ser. No. 60/793,083, filed Apr. 19, 2006.

### BACKGROUND OF THE INVENTION

Many devices have been developed to surgically remove tissue using the ultrasonic vibration of a hollow tube. Kelman (U.S. Pat. No. 3,589,363) first applied the technology using both irrigation over a tubular tip and aspiration applied to the bore of the tube. His method was successfully applied to extraction of cataracts and is now universally used for this purpose in a surgical procedure known as phaco-emulsification. Wuchinich (U.S. Pat. No. 4,223,676) extended the technology to the removal of tissue, such as brain neoplasms and other tumors, previously resistant to attack from such vibration and then subsequently developed apparatus suitable for use in endoscopic (U.S. Pat. No. 4,750,488) and arthroscopic (U.S. Pat. No. 5,176,677) procedures.

Interest in using the technology to remove vitreous humor from the human eye developed concurrently with the introduction of a device to remove the cataract, but limitations upon the permissible length of the tube and the level of vibration prevented its use in this procedure. The vitreous humor, which is interposed between the lens and the retina, while transparent to light, is subject to various maladies, including macular edema or the presence of blood in the substance, which interferes with visual acuity. In such circumstances, it has been found efficacious to remove that portion of the vitreous so affected. The body eventually replenishes the tissue removed. In 1979, Leitgeb et al published (Ultrasonic vitrectomy—an alternative technique to the presently used mechanical procedures, Leitgeb, N, S. Shuy and M. Zirm, Graefes Archiv Ophthalmologie, Albrecht v. Graefes Arch. Klin. Exp. Ophthal., Vol. 209, pp. 263-268) a report of the apparent successful liquefaction of the vitreous humor by a solid stylus vibrating at a frequency of 60 kHz. Their experiments did not seek extraction of the liquefied tissue as it was believed that, as the transformation produced a liquid, ordinary human biological maintenance would ensure its removal and replacement by tissue. In 1980 Kossovskii and Stoliarenko (Case of successful ultrasonic in Terson's Syndrome, Kossovskii, L. V., Stoliarenko, G. E., Vestn. Oftalmol., 1980, No 5, September-October p. 37-9.) published a report of the successful ultrasonic removal of hemorraghically damaged vitreous, using undescribed equipment operating at 40 kHz. Balamuth (U.S. Pat. No. 3,526,219) developed in 1970 devices that used solid vibrating structures to remove tissue by fragmentation and aspiration of the fragmented particles through the interspace between the stylus and a hollow tube surrounding the tip, indicating the feasibility of an alternative form of aspiration that did not require a hollow vibrating tube.

Heretofore, despite Leitgeb and Balamuth's investigations and instrument development, no ultrasonic device has been developed nor gained acceptance to ultrasonically extract vitreous. The diameter and length of a slender stylus that will sustain axial (a direction parallel to the length dimension of the stylus) ultrasonic vibration along its length of the magnitude necessary to cause liquefaction has eluded practitioners. Thus, contemporary technology does not employ ultrasonic vibration, using instead the sonic vibration of a tip traveling inside a hollow tube provisioned with a hole in its wall near the end of the tip.

In known vitrectomy procedures vitreous tissue, a gelatinous-like tissue, is forced partially into a hole by hydrostatic pressure applied to the vitreous humor through hypodermic infusion of fluid into the vitreous compartment of the eye. The vibrating tip repeatedly slices off the tissue protruding through the hole and vacuum applied to the tube draws the tissue so dissected into a receptacle.

### SUMMARY OF THE INVENTION

An object of this invention is to provide an ultrasonic vitreous liquefaction method and apparatus.

A further object of this invention is to provide such an ultrasonic vitreous liquefaction method and apparatus which uses a tapered ultrasonic tip.

In accordance with this invention the tapered ultrasonic tip is solid and is preferably at least partially surrounded by a sheath. The apparatus could be used by first applying irrigation, then aspiration and then ultrasonic vibration.

### THE DRAWINGS

FIG. 1 is a cross-sectional view in elevation of an ultrasonic vitreous liquefaction apparatus in accordance with this invention;

FIG. 2 is an elevational view of an ultrasonic tip having a Gaussian taper which could be usable in the apparatus of FIG. 1;

FIG. 3 is a side elevational view in section of an ultrasonic tip extending through a sheath in accordance with this invention; and

FIG. 4 is a side elevational view of a modified form of ultrasonic tip completely surrounded by a sheet in accordance with this invention.

### DETAILED DESCRIPTION

As has been demonstrated with phaco-emulsification, ultrasonic technology, however, promises the advantage of producing less trauma in the eye than conventional methods and, given both Balamuth's and Leitgeb's work, appears to be possible if unique ultrasonic design innovations are brought to bear upon the problem. In particular, experiments by the inventor have shown that if a solid metal stylus, surrounded by a hollow tube, can be developed that is at least 30 millimeters in length and no more than 0.7 millimeters in diameter and capable of sustaining an excursion at its point of tissue contact of at least 50 microns, then surgical liquefaction and aspiration of vitreous in-vivo is feasible. In general, the apparatus of this invention, as will later be described in detail, includes a transducer for generating an ultrasonic vibration that is communicated to a removable tip. The tip itself is solid and is at least partially surrounded by a stationary sheath that is removable, but in use forms part of the transducer housing. When used for vitrectomy the tip would be inserted into the eye replacing a conventional vitrectomy cutter. Otherwise, all other conventional instrumentation could be used and remains operational. The tip is vibrated, liquefies the vitreous in the region immediately about the free end of the tip and vacuum applied to the interspace between the tip and the sheath removes the vitreous so liquefied from the operative site.

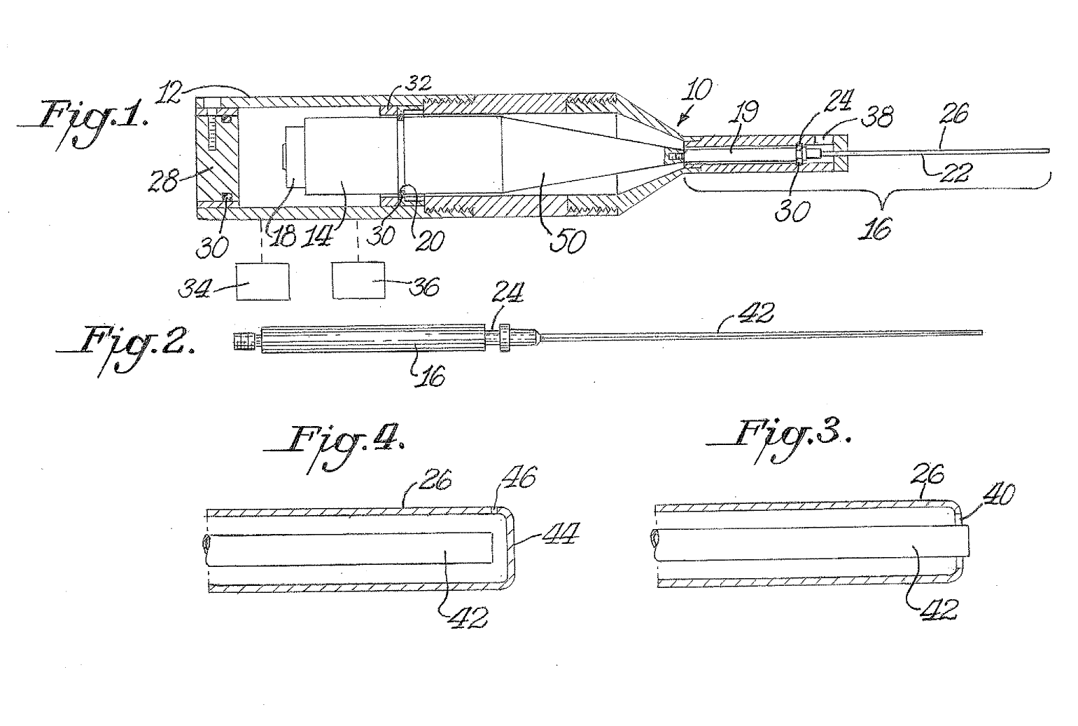

FIG. 1 illustrates an apparatus 10 in accordance with one practice of this invention. As shown therein, apparatus 10 includes a housing 12 containing any suitable transducer 14 which generates an ultrasonic vibration that is communicated to a removable tip 16 as later described.

The transducer 14 shown in FIG. 1 can be of conventional and well established design known in the art, derivative of a Langevin sandwich, named after its inventor, who produced the first known such structure in 1917, using either disks or a tube made of piezo-active material that changes its length is response to the application of an electric or magnetic field. The particular configuration shown here is a refinement invented by Shoh (U.S. Pat. No. 3,524,085) to improve mechanical integrity under substantial vibration. The material is subjected to quiescent compression provided by a pre-stressing compression nut 18 so that the parts of the mechanical assembly remain in intimate contact during vibration. The length of piezo-active material and its associated pre-stressing components comprise at the design frequency a one quarter wavelength section 19 with a node 20 of motion located as indicated in FIG. 1. A second quarter wavelength section 50 is integrally joined to the assembly at the node 20 and serves to amplify somewhat the excursion produced by the transducer during vibration. The transducer, including the piezo-active material, prestressing components and quarter wavelength motion amplifying section 50 comprise a composite rod designed to resonate at a predetermined frequency such that its ends move in opposite axial direction with a point of no motion located at the node 20.

Attached to the end of the amplifying section is a second half wavelength resonator which further amplifies the end motion of the quarter wavelength section of the transducer. This second resonator is designed as a variant of the common stepped horn described by Balamuth in 1954 and comprises an integral assembly of two quarter wavelength sections 19 and 26, having a node 24. (Trans. IRE PGUE-2, (November 1954), p. 23) Such resonators are typically made of a prismatic solid having two different cross sectional areas, one for the first quarter wavelength and the other for the second quarter wavelength section. As such, motion imparted at the resonant frequency to the first section is amplified by the second according to the relation

S₁/S₂

where the subscripts 1 and 2 refer to the first and second sections respectively [of the second quarter+quarter wavelength resonator]. The resonant frequency of such a structure remains the simple expression for that of a uniform rod:

$$f = \frac{c}{2 l}$$

where f is the frequency, c the extensional sound velocity in the material and l the rod's overall length.

The second section is the surgical implement of the invention. It is this section, enclosed in a stationary sheath 26 and exposed to tissue only at its terminus that is inserted into the eye, replacing the conventional vitrectomy cutter. The sheath is removably fastened to the handpiece housing 10 and contacts the second section of the second resonator through an O ring 30 located at the node 24. As Equation 2 reveals, the length of the second resonator is a function of frequency, but, in general, the length of any resonator, including such composite constructions as the transducer, is also inversely proportional to the resonant frequency. Vitrectomies are performed today with the assistance of an operating microscope and the procedure itself is termed micro-surgical. Dexterity is essential and ease of manipulation of the instruments essential for successful extraction of the vitreous humor. Such requirements dictate that the instrument, which in this case is the handpiece, be kept small enough to fit within the span of, and be easily controlled by, the surgeon's fingers. If this span is therefore limited to a 6 or 7 centimeters, then, using titanium which is a metal of which most surgical ultrasonic components are made and which has a sound velocity of about 500,000 centimeter per second, Equation 2 dictates that the resonant frequency must be at least 40,000 Hz.

However, if the tip 16 is made as a simple stepped titanium resonator the length of the second section (and the first for that matter) will be 31 millimeters. The human anatomy requires at least such a length to reach all parts of the vitreous compartment and when other considerations are taken into account, such as the fittings of the protective sheath about the tip and seals for aspiration, a length significantly less that 31 mm will be available for surgical use. It is therefore of interest to use a design that offers a greater length for the second section.

Of interest, as well, is a limitation upon the maximum excursion that titanium can withstand at any given resonant frequency. It has been found when using ultrasonic surgical devices that tissue excision requires a minimum velocity of the tip's free and tissue contacting end and that, in general, the greater this velocity beyond the minimum value needed the greater is the rate at which tissue is excised. At any given specified resonant frequency, the tip excursion, which is the total of the back and forth movement of the tip, is related to the frequency as:

s=vₒ/πf

where s is the excursion and vo is the tip's peak free end velocity. It can be shown that for a rod of constant cross sectional area the peak stress occurs at the node of motion, which is located at the center of the rod, and that it is related to free end excursion as

σ=ρcv₀

where σ is the stress, ρ is the density of the material, c the extensional sound velocity and the free end velocity.

There is a limit to value of σ for all materials. In particular, it has been found that titanium can indefinitely withstand stresses that cyclically vary from tensile to compressive up to 275 Mpa (40,000 pounds per square inch). Hence, there is a definite limit to the value of vₒ and, by Equation 3, to s. Equation 4 also applies to a quarter+quarter wavelength stepped horn. For titanium then, with a density of 4430 Kg/m³, an extensional sound velocity of 5080 m/s and a frequency of 40 kHz, the maximum excursion s, can be computed to be about 100 microns. To further increase this excursion a departure in design must be taken that modifies the shape of the second quarter wavelength section.

For a quarter wavelength resonator having a uniform cross sectional area, the stress diminishes from a maximum value, σₘₐₓ, occurring at the node, until it vanishes at the free end according to the relation

$$σ = {σ}_{max} Cos ( \frac{π x}{2 l} )$$

where x is the distance measured from the node to a point on the section.

It is possible, however, to progressively change the cross sectional area of the second quarter wavelength section of the tip such that the stress remains constant throughout the length. Such an alteration allows the length of the second section to be made indefinitely long and to be capable of indefinitely large excursions, although, in practice, the cross sectional area becomes at some point so small that it defies fabrication or the portion of the tip so affected becomes too fragile to sustain normal use in surgical procedures. A section having the property just described is known as a Gaussian tip as the diminution in cross section area contains the Gaussian mathematical function. Such Gaussian tip 16 is shown in FIG. 2. Wuchinich has described the design of such resonators for use in endoscopic surgical applications and his discussion applies equally well to the present invention.

To extend the length of the second section of the vitrectomy tip so that it becomes long enough for use in reaching the entire vitreous compartment of the eye in handpieces operating at frequencies extending from 40 kHz, a portion of the tip is tapered to produce constant stress until the overall length of the section exceeds 30 millimeters, with the added benefit of a significantly larger permissible excursion than is possible using a section having a uniform cross sectional area.

It is also possible, without resorting to a Gaussian tapered tip, to achieve the needed tip length by simply tapering the tip uniformly. Such a design will not provide, in its optimal configuration, as much length or permissible excursion as a tip having an optimal Gaussian taper, but the motion and length may nevertheless be entirely adequate for surgical purposes.

Referring again to FIG. 1 the apparatus 10 includes a plug 28 at the end of the housing 12. Housing 12 is formed in multiple sections which are mounted together in any suitable fashion, such as by threaded connections as illustrated. As illustrated the housing 12 is formed of three pieces offset 1200. Suitable O-rings or other sealing devices 30 are also appropriately located in apparatus 10. A squeeze ring 32 is located at the transducer's node 20. Apparatus 10 may also be provided with any suitable irrigation structure schematically illustrated by the block with the reference numeral 34 and any suitable aspiration structure schematically illustrated by the block with the reference numeral 36. An aspiration tubing access 38 is also shown.

FIG. 3 illustrates in greater detail the sheath 26 which has an opening 40 at its free end through which the free end 42 of the tip 16 extends. FIG. 3 illustrates a variation of the invention where the free end 42 is of uniform diameter rather than being tapered.

FIG. 4 illustrates a further variation of the invention wherein the sheath 26 has a closed end 44. A side cutting port 46 is located in the peripheral wall of sheath 26 generally located at the end portion of the free end 42 of the tip. An advantage of the arrangement shown in FIG. 4 is that by using a liquefaction tip in a sheath with a closed end damage to underlying tissue is prevented, such as damage to the retina while excavating underlying tissue.

The sheath 26 is a separate component part which is detachably or removably mounted to the housing 12. The sheath 26 surrounds the second quarter wavelength which is the second half of the second resonator as illustrated in FIG. 1. The first resonator is the transducer. Together the first and second half of the second form the tip 16. The free end of the tip could be tapered such as having a Gaussian taper as illustrated in FIG. 2 or could be of uniform diameter such as the free ends 42 illustrated in FIGS. 3-4. The sheath 26 could entirely enclose or surround the free end of the tip as shown in FIG. 4 wherein there is a side port 46 just below the extreme end of the tip to admit tissue for liquefaction by the end of the tip. Alternatively, as shown in FIG. 3 the sheath 26 could be open ended without a side port with the outer end of the tip ending just within the open end of the sheath or flush with the sheath opening 40 or slightly protruding from the sheath as shown in FIG. 3.

In operation, particularly for vitrectomy tissue liquefaction would be achieved by first applying irrigation and then aspiration and then ultrasonic vibration.

Among the features of the invention is that a solid ultrasonic tip is used for tissue liquefaction. The ultrasonic tip could be tapered and could be a Gaussian taper. The liquefaction could be obtained using irrigation and/or aspiration. These various alternatives could be used with pulsed on-off ultrasonic vibration, the pulse rate being of a lower frequency than the frequency of vibration.

While the invention has been particularly described with regard to vitrectomy, it is to be understood that the invention is not limited to that specific application, but could also be used where appropriate for liquefying tissue or for other suitable uses.

It is further to be understood that where reference has been made to various patents and publications, all of the details of those patents and publications as well as the details of the provisional application on which this application is based, are incorporated herein by reference thereto.

What is claimed is:

1. An ultrasonic liquefaction apparatus comprising an implement having a transducer for generating ultrasonic vibration, a removable tip operatively connected to said transducer for being longitudinally vibrated by said transducer, and said tip being a solid ultrasonic tip.

2. The apparatus of claim 1 including a housing, said transducer being mounted in said housing, said ultrasonic tip extending longitudinally outwardly from said housing, and said ultrasonic tip being at least partially surrounded by a stationary sheath mounted to said housing.

3. The apparatus of claim 2 wherein said ultrasonic tip is tapered.

4. The apparatus of claim 2 wherein said ultrasonic tip is a Gaussian tapered tip.

5. The apparatus of claim 2 including aspiration structure communicating with said housing.

6. The apparatus of claim 2 including irrigation structure communicating with said housing.

7. The apparatus of claim 6 including aspiration structure communicating with said housing.

8. The apparatus of claim 2 wherein said sheath is removably mounted to said housing and partially surrounds said tip, and part of said tip extending longitudinally outwardly from said sheath and being exposed.

9. The apparatus of claim 2 wherein the portion of said tip extending longitudinally outwardly from said housing is entirely surrounded by said sheath, and said sheath being removably mounted to said housing.

10. The apparatus of claim 9 wherein said sheath has a closed end and a peripheral side wall, and a side cutting port being in said peripheral side wall.

11. The apparatus of claim 10 wherein said tip terminates in a free end, and said side cutting port being located at said free end of said tip.

12. A method of ultrasonic liquefaction comprising providing an implement having a transducer and a solid ultrasonic tip, creating ultrasonic vibration from the transducer, transmitting the ultrasonic vibration to the solid tip to longitudinally vibrate the solid tip, surrounding at least a portion of the solid tip by a sheath, and inserting the solid tip into an operative site to create tissue liquefaction at the operative site.

13. The method of claim 12 including first applying irrigation and then aspiration and then ultrasonic vibration to obtain the liquefaction.

14. The method of claim 12 including providing the solid tip with a Gaussian taper.

15. The method of claim 12 including mounting the free end of the tip at least partially within a sheath.

16. The method of claim 12 including surrounding the entire free end of the tip within a sheath.

17. The method of claim 16 including providing the sheath with a closed end and with a side cutting port in the peripheral side wall of the sheath located at the free end of the tip.

18. The method of claim 12 including using pulsed on/off ultrasonic vibration with the pulse rate being of a lower frequency than the frequency of vibration.

19. The method of claim 12 wherein the liquefaction is of vitreous tissue.

20. The method of claim 12 wherein the liquefaction is used to remove vitreous humor from the human eye.
